(19) United States
(12) Patent Application Publication
(10) Pub. No.: US 2012/0029652 A1
(43) Pub. Date: Feb. 2, 2012

(54) APPARATUSES AND METHODS FOR IMPLANTING GASTROINTESTINAL STENTS

(76) Inventor: Mihir S. Wagh, Gainesville, FL (US)

(21) Appl. No.: 13/260,853

(22) PCT Filed: Apr. 1, 2010
(86) PCT No.: PCT/US2010/029557

§ 371 Date: Sep. 28, 2011

Related U.S. Application Data
(60) Provisional application No. 61/165,599, filed on Apr. 1, 2009.

(51) Int. Cl. A61F 2/04

(52) U.S. Cl. 623/23.7

(57) ABSTRACT

In one embodiment, a surgical device includes a stent deployment device that includes a stent, a dilator, and a cutting element that can be selectively extended from and retracted into the surgical device. In some embodiments, the surgical device can be used to implant a stent in a patient by passing the surgical device through a natural orifice of the patient, accessing the peritoneal cavity with the surgical device, translumenally accessing the lumen of the small intestine with the surgical device, passing a stent deployment device into the lumen with the surgical device, and expanding the stent within the lumen using the surgical device. In other embodiments, the surgical device can be used to perform endoscopic translumenal pancreatic pseudocyst drainage.

### CROSS-REFERENCE TO RELATED APPLICATION

This application claims priority to copending U.S. provisional application entitled, “Apparatuses And Methods For Implanting Gastrointestinal Stents,” having Ser. No. 61/165,599, filed Apr. 1, 2009, which is entirely incorporated herein by reference.

### BACKGROUND

Stents are used in various lumens within the body. For example, it is now common to implant stents within the coronary arteries during angioplasty procedures. Stents are now also used within the digestive system. For example, stents are implanted within the esophagus, duodenum, bile duct, pancreatic duct, and the colon.

There are other lumens within the body in which stents could be useful. For example, stents could be placed within the small intestine to correct gastrointestinal obstructions or fistulas. At present, however, small intestine stent implantation is not routinely performed due to the difficulty in achieving endoscopic access to the small intestine via enteroscopy.

### BRIEF DESCRIPTION OF THE FIGURES

The present disclosure may be better understood with reference to the following figures. In the figures, like reference numerals designate corresponding parts throughout the figures, which are not necessarily drawn to scale.

FIGS. 1A-1E are schematic views of a patient and his digestive system, and illustrate steps of a small intestine stent implantation procedure performed via a natural orifice.

FIGS. 2A and 2B are side and end views, respectively, of a first embodiment of an integrated surgical device that can be used in the procedure illustrated in FIGS. 1A-1E.

FIGS. 3A-3H are schematic views of translumenal access of the small intestine for purposes of stent deployment using the surgical device shown in FIGS. 2A and 2B.

FIGS. 4A and 4B are side and end views, respectively, of a second embodiment of an integrated surgical device that can be used in the procedure illustrated in FIGS. 1A-1E.

### DETAILED DESCRIPTION

Disclosed herein are apparatuses and methods for implanting a stent within the gastrointestinal tract. More particularly, disclosed are apparatuses and methods for implanting a stent within the small intestine, also referred to as the small bowel. In the disclosed methods, the small intestine is accessed using a natural orifice as an entry point. In some embodiments, the peritoneal cavity is first accessed via the stomach, colon, or vagina and the small intestine is then entered translumenally. In some embodiments, entry into the small intestine and deployment of the stent therein is achieved using a single integrated surgical device that includes a cutting element, a guidewire, a dilator, and a stent deployment device. Although particular embodiments of apparatuses and methods are disclosed herein, it is noted that those embodiments are mere implementations of the disclosed inventions and that alternative embodiments are both possible and intended to fall within the scope of the present disclosure.

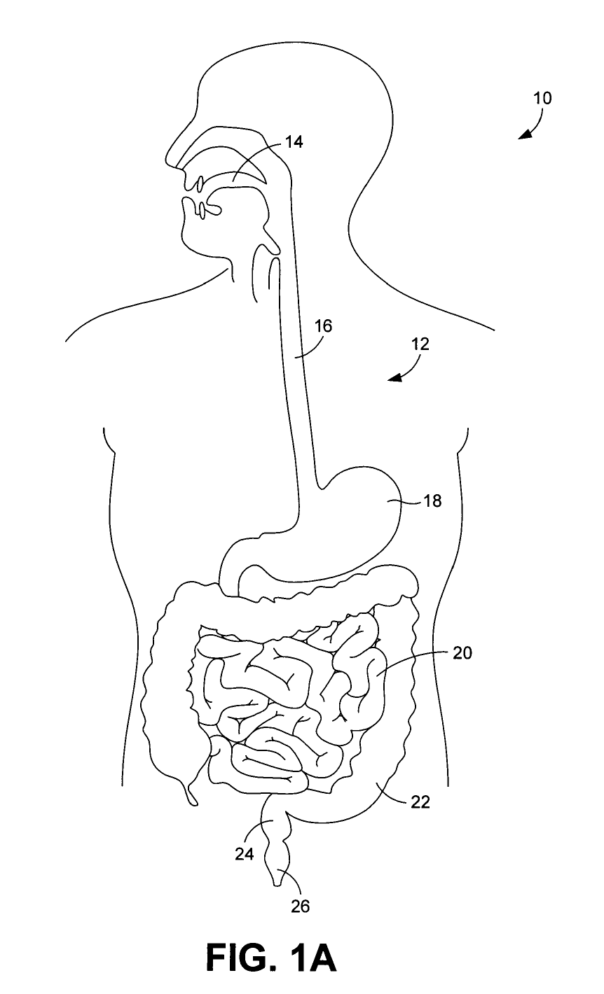

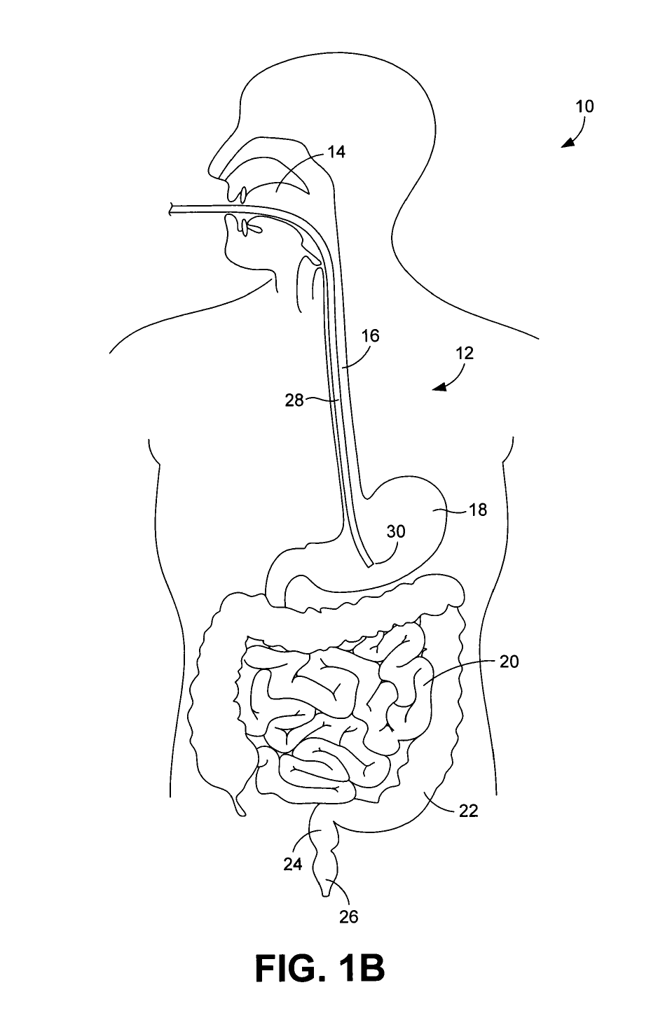

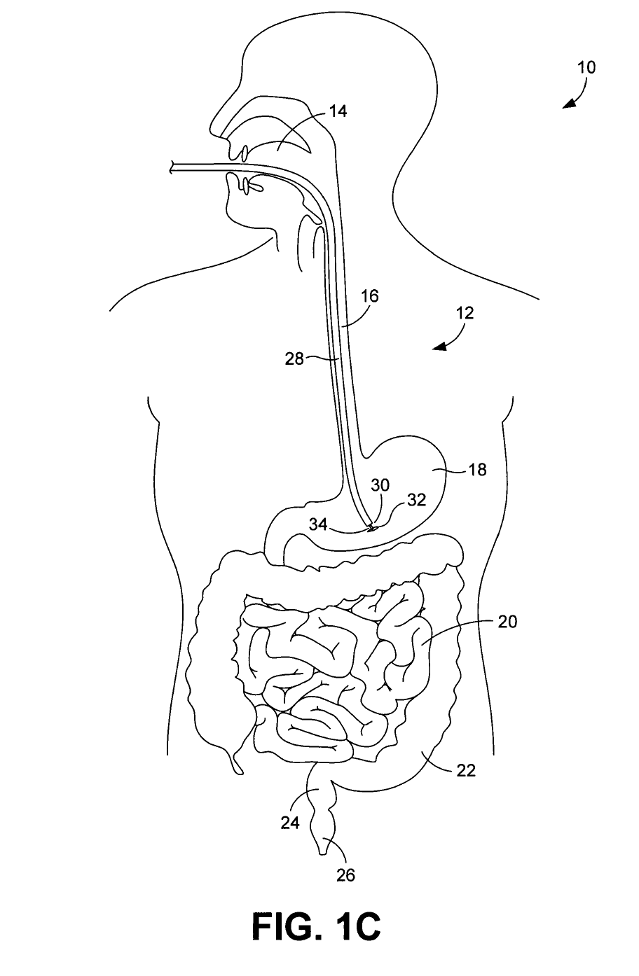

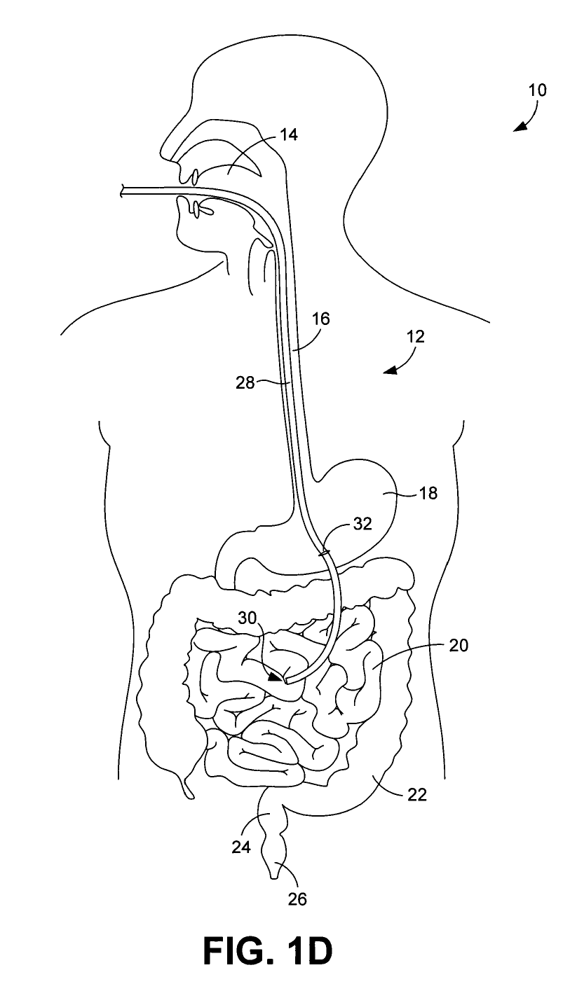

FIG. 1A schematically illustrates a patient 10 and his digestive system 12, including the mouth 14, the esophagus 16, the stomach 18, the small intestine 20, the large intestine 22, the rectum 24, and the anus 26. For purposes of this disclosure, it is assumed that there is a medical condition, such as an obstruction or fistula (not illustrated) within the small intestine 20, that could be alleviated by stent implantation. To avoid the difficulty of achieving endoscopic access to the small intestine via enteroscopy, transluminal endoscopic surgery is performed using a natural orifice as the point of entry. Such procedures are occasionally referred to as “natural orifice translumenal endoscopic surgery” or “NOTES” procedures. In the example that follows, the natural orifice that is chosen is the mouth (and esophagus). It is noted, however, that another natural orifice could instead be chosen, such as the colon or urethra or, in cases in which the patient is female, the vagina.

Referring next to FIG. 1B, the shaft of an endoscope 28, such as a flexible, manually articulable endoscope, is fed through the mouth 14 and the esophagus 16 until a distal tip 30 of the endoscope reaches the interior of the stomach 18. The anterior wall of the stomach 18 is then identified by endoscopically viewing the indentation produced by external palpitation. With reference to FIG. 1C, a full-thickness gastric incision 32 is made through the stomach wall using an appropriate cutting element 34. In some embodiments, the cutting element 34 comprises an endoscopic cutting element that, as illustrated in FIG. 1C, is passed through a lumen (e.g., working channel) of the endoscope 28 so that the incision can be made under direction endoscopic viewing. By way of example, the cutting element 34 comprises an electrocautery needle knife.

Once the incision 32 has been made, the endoscope 28 is passed through the incision and the stomach wall to achieve access to the peritoneal cavity. Transgastric endoscopic peritoneoscopy is then performed and the small intestine 20 is identified by direct endoscopic visualization. As shown in FIG. 1D, the tip 30 of the endoscope 28 can then be maneuvered to a desired location of the small intestine 20, again using direct endoscopic visualization.

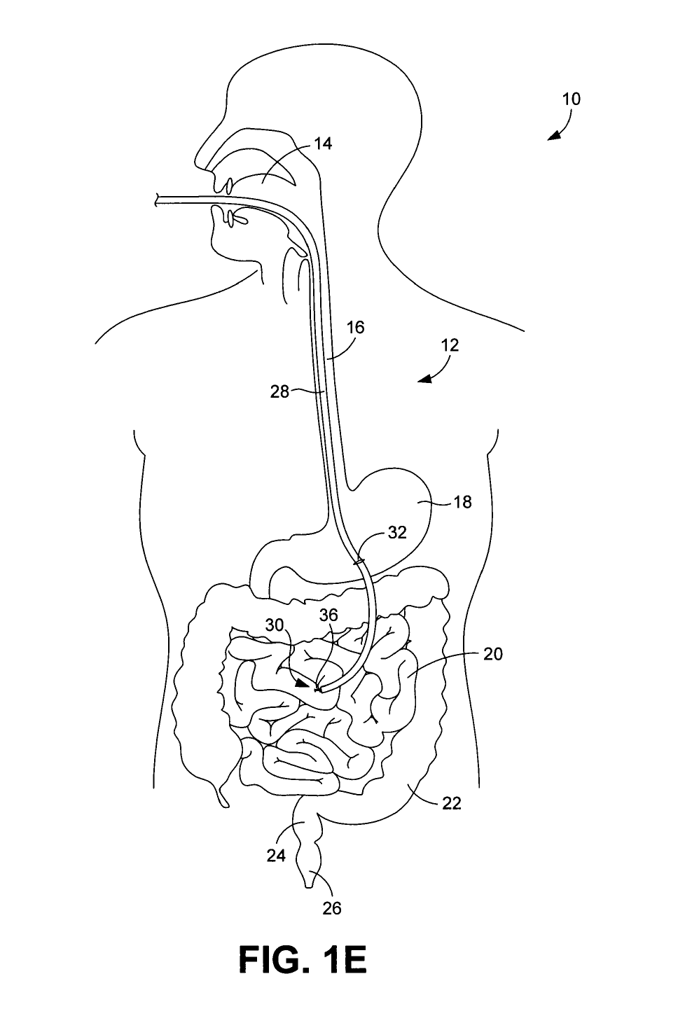

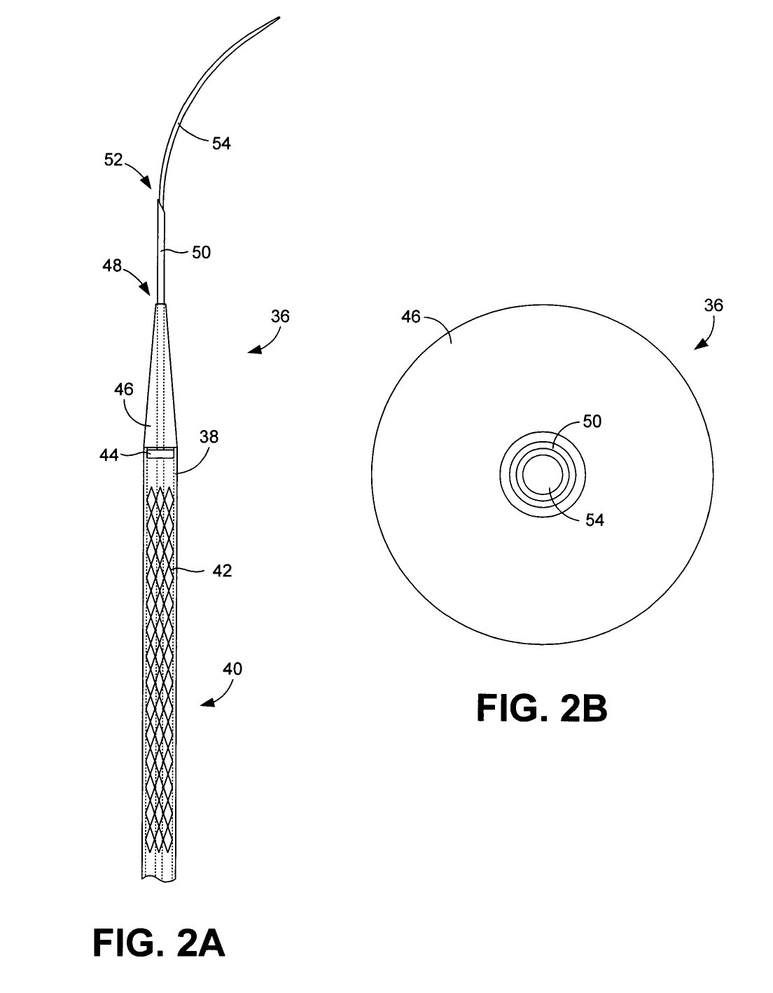

Once the desired entry point of the small intestine 20 has been identified, an enterotomy can be performed to access the lumen of the small intestine, as illustrated in FIG. 1E. In some cases, such access can be achieved using an integrated surgical device specifically designed for use in both accessing the small intestine lumen and deploying a stent within that lumen. FIGS. 2A and 2B illustrate a first embodiment of such a device 36. As indicated in FIG. 2A, the device 36 comprises an elongated flexible tube 38 (only a portion of which visible in the figure) that can be advanced through an endoscope, such as endoscope 28. Comprised by the tube 38 is an integral stent deployment device 40 that includes a stent 42. In some embodiments, the stent 42 comprises an initially-compressed, self-expanding metal stent (SEMS). Also comprised by the tube 38 is one or more radio-opaque markers 44, such as a metal band, that can be used to identify the location and/or orientation of the device 36, and its stent 42, under fluoroscopy.

The integrated surgical device 36 further comprises a dilator 46 whose distal tip 48 forms the distal tip of the device. In the embodiment of FIGS. 2A and 2B, the dilator 46 comprises a graduated frustoconical dilator. As described below, however, alternative types of dilators can be used. Also comprised by the device 36 is a cutting element 50 that can be selectively extended and retracted from the distal tip of the device. The cutting element 50 is shown in the extended orientation in FIG. 2A. In some embodiments, the cutting element 50 comprises an electrocautery needle knife. Indeed, the cutting element 50 can, in some cases, be the electrocautery needle knife that was used to incise the stomach wall (see FIG. 1C). As shown in FIG. 2A, the cutting element 50 comprises a sharp, beveled tip 52.

The integrated surgical device 36 also includes a guidewire 54 that can be extended from the distal tip of the device. In the embodiment illustrated in FIGS. 2A and 2B, the guidewire 54 is positioned within an inner lumen of the hollow cutting element 50 and therefore can be extended from the tip 52 of the cutting element. In alternative embodiments, however, the guidewire 54 need not be contained with the cutting element 50 (e.g., if the guidewire is too large to be passed through the cutting element or the cutting element is not hollow). In such a case, the guidewire 54 can be passed through an alternative lumen of the device 36 (not shown). Regardless, the guidewire 54 can comprise a coated steel and/or shape-memory alloy (e.g., nickel titanium) wire and a very flexible or “floppy” tip that facilitates traversal of the small intestine. In at least some embodiments, the guidewire 54 is preloaded within the device 36 to reduce the time necessary to perform the implantation procedure.

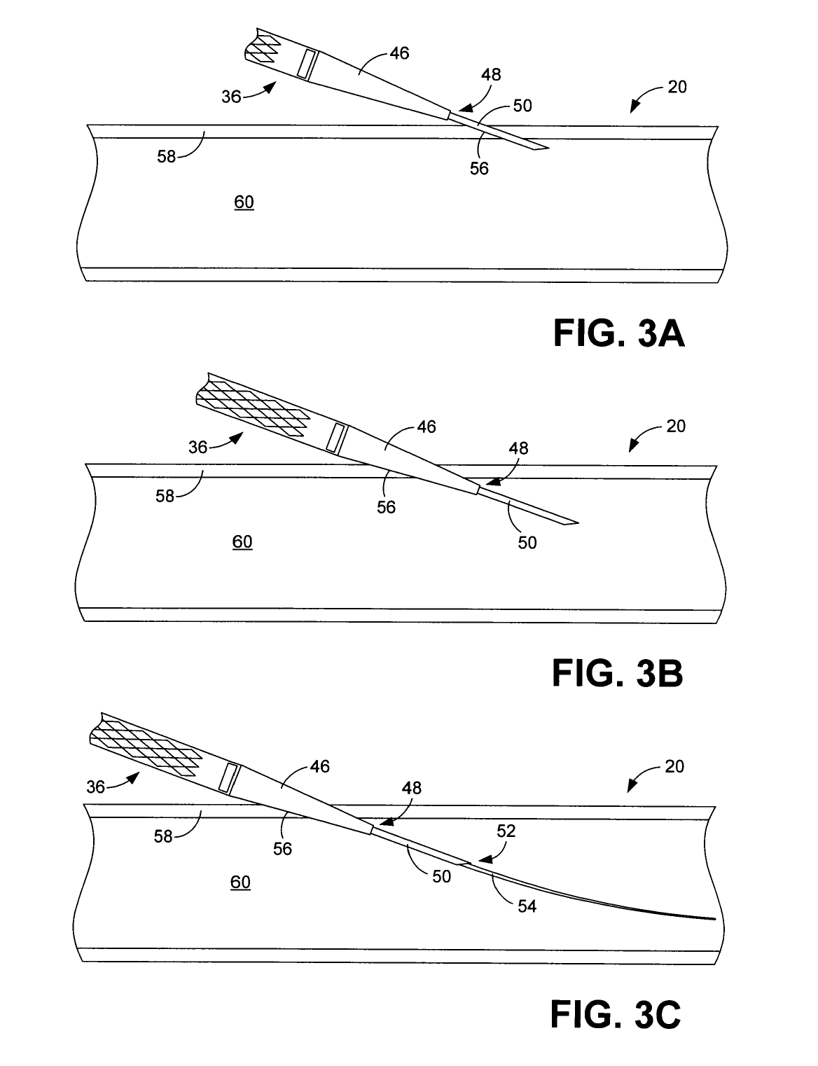

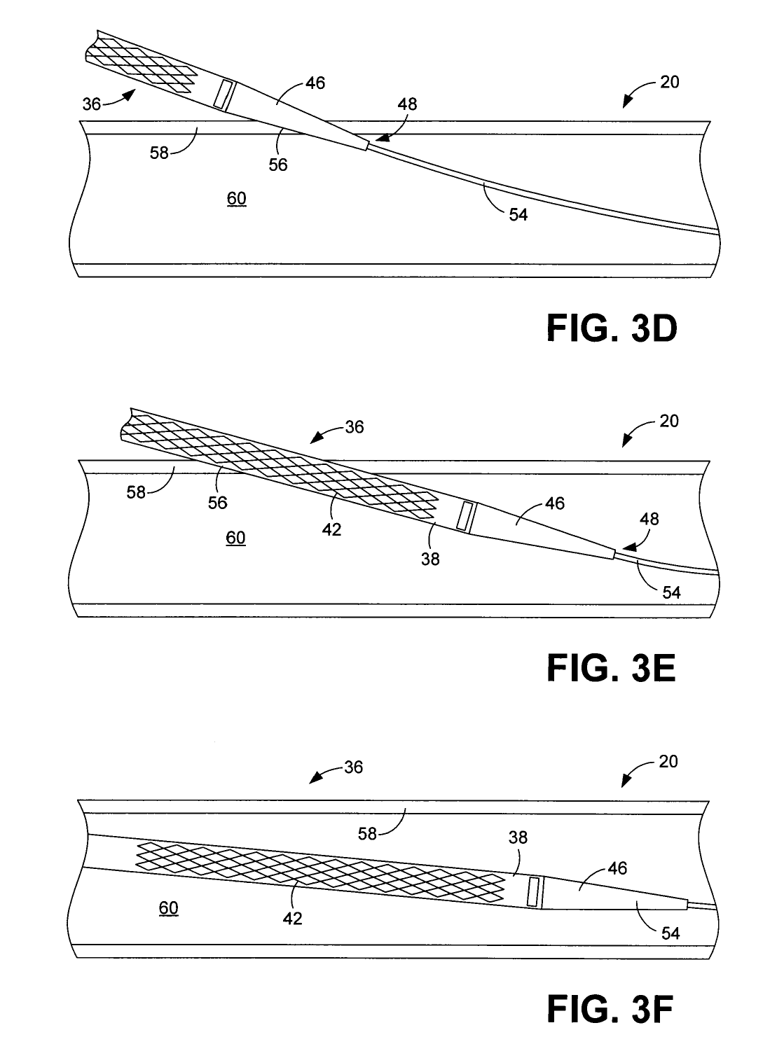

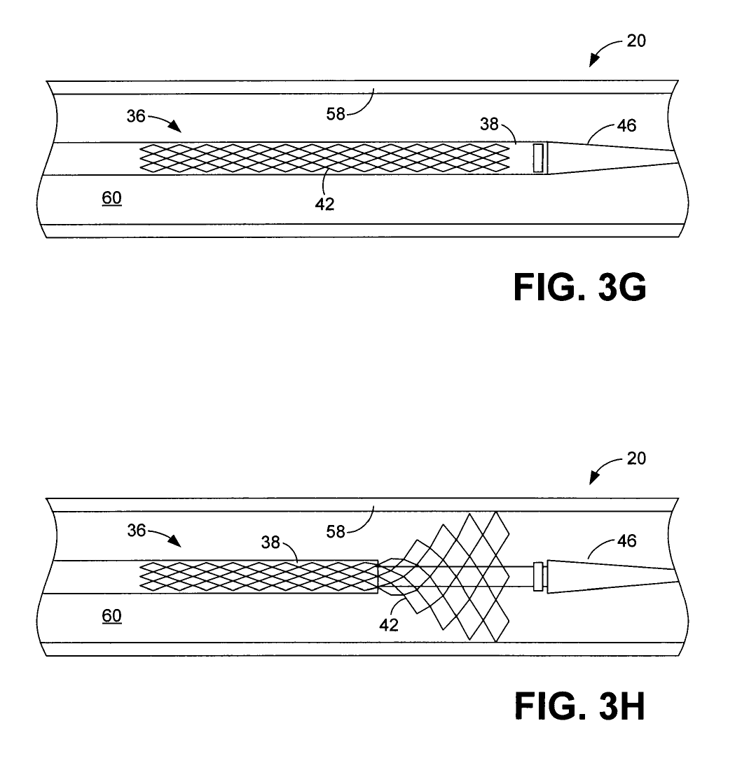

FIGS. 3A-3F illustrate an example of use of the integrated surgical device 36 in preparation for stent deployment within a section of the small intestine 20. Beginning with FIG. 3A, the cutting element 50 is extended from the distal tip 48 of the device 36 and an electric current is applied to the element so that the element can easily cut a hole 56 through the wall 58 of the small intestine 20. Once the hole 56 has been formed, the tip 48 of the device 36 can be passed through the hole and into the lumen 60 of the small intestine 20, as shown in FIG. 3B (see also FIG. 1E). Notably, the size of the hole 56 is increased by the dilator 46 as the tip 48 is passed deeper into the lumen 60.

Referring next to FIG. 3C, the guidewire 54 is extended from the device 36 (e.g., from the tip 52 of the cutting element 50) and into the lumen 60. As soon as a desired length of the guidewire 54 has been positioned within the lumen 60, the cutting element 50 is retracted, as indicated in FIG. 3D, to avoid unintended laceration of the small intestine wall 58. Next, the device 36 is further advanced through the hole 56, as shown in FIGS. 3E and 3F, until the stent 42 is positioned at an appropriate location for deployment. If necessary, bowel loops can be manipulated with endoscopic forceps at any time during the procedure (not shown). At this point, the device 36 remains in the hole 56, although its passage through the hole is not visible in FIG. 3F due to the advancement of the device through the lumen 60. Once the integrated surgical device 36 is in the desired position, such as the position shown in FIG. 3G, the stent 42 can be deployed within the small intestine lumen. By way of example, the elongated tube 38, which serves as a sheath for the stent 42, can be withdrawn to enable the stent to expand, as is schematically shown in the FIG. 3H. Alternatively, the stent 42 can be advanced out from the tube 38 (not shown).

Assuming the stent 42 has been correctly deployed in the desired position within the small intestine 20, the integrated surgical device 36 can be withdrawn, and the hole 56 that was formed in the small intestine can be closed. In some embodiments, the hole 56 can be closed using endoscopic clips. In other embodiments, the hole 56 can be sutured using a suitable endoscopic suturing device. After the hole 56 has been closed, the tip 30 of the endoscope 28 can be withdrawn back into the stomach 18 (FIG. 1B), and the stomach incision 32 can also be closed.

As can be appreciated from the foregoing description, a stent can be implanted within the small intestine using transluminal endoscopic surgery with entry via a natural orifice, thereby avoiding the need to perform small intestine enteroscopy or open surgery. When an integrated surgical device is used, time is saved because there is no need to exchange a cutting tool for a stent deployment device. In addition, because the cutting element and the stent deployment device are integrated into a single device, the size of the hole that must be made through the wall of the small intestine can be smaller, thereby facilitating easier closure and reducing patient risk.

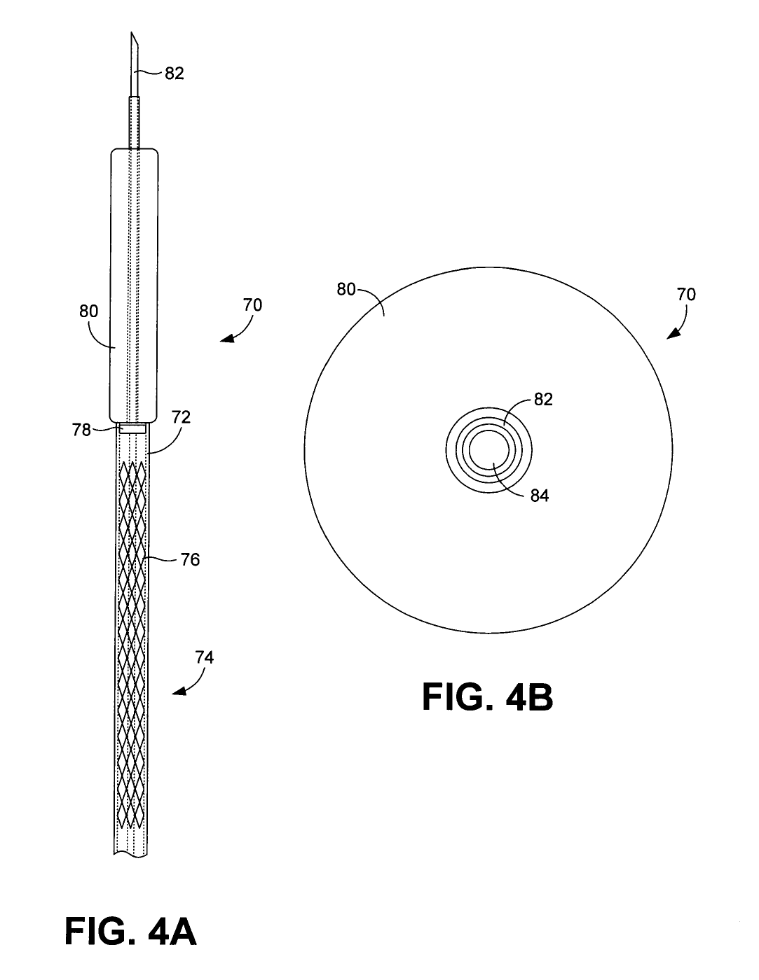

FIGS. 4A and 4B illustrate a second embodiment of an integrated surgical device 70 that can be used in the above-described procedure. The device 70 is similar in many ways to the device 36 shown in FIGS. 2A and 2B. Therefore, as indicated in FIG. 4A, the device 70 comprises an elongated flexible tube 72 (only a portion of which visible in the figure) that contains an integral stent deployment device 74, which includes a stent 76, and one or more radio-opaque makers 78. The device 70 also includes a dilator 80. However, unlike dilator 46, the dilator 80 comprises an inflatable balloon dilator that can be expanded from an initial compressed orientation (not shown) when filled with an appropriate fluid (e.g., sterile water). The expanded state is illustrated in FIGS. 4A and 4B. In addition, the device 70 includes an extendible/retractable cutting element 82 as well as an extendible guidewire 84 (FIG. 4B).

The integrated surgical device 70 can be used in similar manner to the integrated surgical device 36. The primary difference between the two devices is that, with the device 70, dilation of the hole formed by the cutting element 82 is achieved by expanding the balloon of the dilator 80 as opposed to urging a graduated dilator through the hole.

Although the disclosed surgical devices have been described as being well suited for transluminal endoscopic surgery and gastrointestinal stent placement, it is noted that the devices can be used for other purposes. For example, the surgical devices can be used in pancreatic pseudocyst drainage. In such a case, the device can be used to pass through the wall of the stomach or the small intestine to access a cyst formed on the pancreas. The device can be used to form an incision through the stomach or intestine wall and in the adjacent cyst wall, dilate the hole formed by the incision, place a stent within the pancreas cyst that extends to the stomach or intestine, and allow the cyst to drain into the stomach or small intestine. Accordingly, one device could be used to perform the tasks now performed by three independent devices. The stent can be left in place for a few weeks until the cyst is fully drained. At that point, the stent can be endoscopically removed.

1. A surgical device comprising:
a stent deployment device that includes a stent;
a dilator; and
a cutting element that can be selectively extended from and retracted into the surgical device.

2. The surgical device of claim 1, further comprising an elongated flexible tube and wherein the stent deployment device is housed within the elongated flexible tube.

3. The surgical device of claim 1, wherein stent is a self-expanding stent.

4. The surgical device of claim 1, wherein the dilator is positioned distal to the stent deployment device.

5. The surgical device of claim 1, wherein the dilator forms a distal tip of the surgical device.

6. The surgical device of claim 5, wherein the cutting element is extendible from and retractable into the distal tip of the dilator.

7. The surgical device of claim 1, wherein the dilator comprises a graduated frustoconical dilator.

8. The surgical device of claim 1, wherein the dilator comprises an expandable balloon.

9. The surgical device of claim 1, wherein the cutting element comprises an electrocautery needle knife.

10. The surgical device of claim 1, further comprising a guidewire that is extendable from the surgical device.

11. The surgical device of claim 10, wherein the guidewire is extendable from a tip of the cutting element.

12. A method for implanting a stent in a patient using a single surgical device, the method comprising:
passing the surgical device through a natural orifice of the patient;
accessing the peritoneal cavity with the surgical device;
translumenally accessing the lumen of the small intestine with the surgical device;
passing a stent deployment device into the lumen with the surgical device; and
expanding the stent within the lumen using the surgical device.

13. The method of claim 12, wherein the natural orifice comprises the mouth and wherein accessing the peritoneal cavity comprises inserting the surgical device into an endoscope and passing the endoscope through the mouth and the esophagus and into the stomach, and forming an incision through the stomach wall with a cutting element of the surgical device.

14. The method of claim 12, wherein the natural orifice comprises the anus and wherein accessing the peritoneal cavity comprises inserting the surgical device into an endoscope and passing the endoscope through the rectum and into the colon, and forming an incision through the colon wall with a cutting element of the surgical device.

15. The method of claim 12, wherein the natural orifice comprises the urethra and wherein accessing the peritoneal cavity comprises inserting the surgical device into an endoscope and passing an endoscope through the urethra and into the urinary bladder, and forming an incision through the bladder wall with a cutting element of the surgical device.

16. The method of claim 12, wherein the natural orifice comprises the vagina and wherein accessing the peritoneal cavity comprises inserting the surgical device into an endoscope and passing an endoscope through the vagina, and forming an incision through the vagina wall with a cutting element of the surgical device.

17. The method of claim 12, wherein translumenally accessing the lumen of the small intestine comprises cutting a hole through the wall of the small intestine using a cutting element of the surgical device.

18. The method of claim 17, further comprising passing the tip of the surgical device into the small intestine lumen.

19. The method of claim 18, further comprising extending a guidewire from the surgical device and into the small intestine lumen.

20. The method of claim 19, further comprising retracting the cutting element into the surgical device.

21. The method of claim 20, further comprising passing the surgical device into the small intestine lumen at least to an extent at which a stent deployment device of the surgical device is contained within the lumen.

22. The method of claim 21, further comprising deploying the stent within the small intestine lumen.
